(19) United States
(12) Patent Application Publication
(10) Pub. No.: US 2007/0072250 A1
(43) Pub. Date: Mar. 29, 2007

(54) Method and system for analysis of cancer biomarkers using proteome image mining

(75) Inventors: Chul-Woo Kim, Gyeonggi-do (KR); Young-Mee Park, Seoul (KR); Jong-Sou Park, Gyeonggi-do (KR); Sung-Do Chi, Gyeonggi-do (KR); Syng-Yup Ohn, Seoul (KR); Soo-Chan Hwang, Gyeonggi-do (KR)

Correspondence Address: Tanya A Arenson;Medlen & Carroll, 101 Howard Street, Suite 350, San Francisco, CA, 94105, US

(73) Assignee: BIOINFRA INC.

(21) Appl. No.: 10/510,937

(22) PCT Filed: Dec. 24, 2002
(86) PCT No.: PCT/KR02/02427

§ 371 Date: Feb. 3, 2006

(30) Foreign Application Priority Data
Apr. 8, 2002 (KR) 10-2002-0019028
Oct. 31, 2002 (KR) 10-2002-0067298

(51) Int. Cl. G01N 33/574; G06F 19/00

(52) U.S. Cl. 435/7.23; 702/19

(57) ABSTRACT

The present invention provide a system and a method for detection of cancer, by producing a serum proteome standard by an image mining technique, and provide cancer-specific biomarkers. Disclosed is a method of analyzing cancer, comprising the steps of transforming serum proteomes from normal individuals and individuals having cancer into two-dimensional images, and constructing a database consisting of the proteome standard by an image mining technique; inputting a serum proteome from a subject of interest, transforming the serum proteome into a image and comparing the structure of the serum proteome pattern of the subject with the proteome standard and determining whether the serum proteome of the subject is normal or abnormal. The system and method for cancer analysis facilitates detection of cancer by constructing a database from a plurality of serum proteome using an image technique and comparing serum proteome of a subject with a proteome standard.

### TECHNICAL FIELD

The present invention relates to a method of mining of meaningful biomarker spots in a specific disease and diagnostic screening of diseased state by transforming each of the separated states of serum proteins from a plurality of normal and diseased living individual on a 2D(2 dimensional)gel into an image, producing a disease-specific serum proteome standard (proteome pattem) by an image mining technique, and comparing proteome of a subject organism with proteome standards of normal or diseased individuals. The present invention is also concerned with a system introducing a method of screening cancer. More particularly, the present invention relates to a system and a method for early detection of cancer, which are capable of identifyig proteome pattern of a specific cancer by producing serum proteome standards by an image mining technique and then comparing the proteome of a subject with the proteome standards. Further, the present invention relates to a proteome pattern for a specific cancer type, comprising one or more specific serum proteins, which can be used as a cancer-specific biomarkers in such a system or method for cancer diagnosis.

### BACKGROUND ART

Recently, with the rapid development of bioinformatics and analysis techniques of DNA sequences, a large volume of genomic data of humans, animals, plants and microindividuals has become known, thus giving rise to a broad range of industrial applications, including diverse research fields, such as development of new pharmaceutical preparations, new diagnostic tools for diseases and production of genetically modified plants. Bioinformatics, which is a technique of rapidly and effectively processing a large volume of data through fusion of Biotechnology (BT) and information technology (IT), can collect, save and analyze a large volume of information carried by the living individual, apply the resulting data to a wide variety of fields, such as pharmaceuticals, foods, agriculture or environmental engineering, thereby creating high-value products.

As a result of completion of the human genome project and the development of bioinfomatics, it was found that genes play a critical role in determining cause-effect relationship of diseases in humans and phenotypes of humans. That is, in spite of having almost similar DNA sequences, humans show differences in their appearance, height, character, and features of an individual are determined only by his/her human genome if not influenced by the environmental factors.

In this regard, human genome and clinical data obtained using the same can be applied to treat incurable diseases such as cancer, where, in case of cancer, much better therapeutic effects are expected if discovered at the early stage. Urines, tears, saliva, etc., have been used for detection of diseases at the early stage, and recently, serum proteomes are often used.

Multifactorial disease, like cancer, is developed by combinatorial action of genetic factors and environmental factors. For the diagnosis and prognostic evaluation of cancer, overall proteome changes accompanied with cancer development, progression and malignant degeneration of cancer must be analyzed. In case of cancer, influenced by not one or two kinds of abnormal cells or tissues, but by abnormal fimction due to its involvement of several organs, body fluids such as serum are suitable as biological samples capable of indicating changes in proteome. Especially, in case of diseases which are difficult to diagnose in the early stage (and prognose), such as lung cancer, blood serum is considered as an optimal sample because of being easily obtainable and widely used in clinical tests.

When comparing components of a serum proteome of a normal human with that of a cancer patient, protein composition of the serum proteome is predicted to differ. However, at present, the specific differences in protein compositions are unknown. Although the human genomic map was completed by the human genome project, there is still no information precisely identifying the relationship between genes and proteins expressed from the information encoded in the genes.

In particular, diseases such as cancer are induced by specific modifications of specific genes, and such modifications are thought to evoke changes in the protein composition of the serum proteome. Through analysis of such a change of composition of the serum proteome, diseases such as cancer can be discovered at the early stage, as disclosed in the prior art. For example, PCT Application No. PCT/AU01/00877, filed in Jul. 19, 2001 by Rarish, Christopher, Richard, et al., describes reduced or enhanced molecular species found by comparing a profile of molecular species in a serum sample from a human or animal subject having cancer with that in a serum sample from a healthy human or animal subject using a mass spectrometry-based method, and their use as cancer markers. In detail, disclosed is a method of identifying a cancer marker, comprising the steps of (i) separating a blood fraction from a human or animal subject having cancer by mass spectrometry, (ii) separating a blood fraction from a healthy human or animal by mass analysis; and (iii) comparing a profile of molecular species at step (i) with that at step (ii) and identifying increased or reduced molecular species, wherein an increased or reduced level of the molecular species indicates that the molecular species is a cancer marker.

PCT Application No. PCT/US01/28133, filed in Sep. 7, 2001 by Yip, Tai-Tung et al., discloses a novel protein marker for diagnosis of breast cancer, which was discovered using Surface-Enhanced Laser Desorption/Ionization (SEIDi) mass spectrometry, in which a breast cancer patient and a normal human can be distinguished by determining presence or absence, the amount and detected frequency of the protein marker.

Recently, Emanuel F., Petricoin m, et a (Lancet, 359:572-577, 2002) reported a proteome pattern of ovarian cancer patients, which is obtained using Surface-Enhanced Laser Desorption/Ionization-Time of Flight (SELDI-TOF) mass spectrometry and differs from that of normal humans, and such a proteome pattern can be applied for diagnosis of ovarian cancer with high sensitivity and specificity.

However, all of the above-mentioned research utilized SELDI-TOF or MALDI-TOF mass spectrometry, which is a one-dimensional analysis pattern, to find cancer-specific serum molecular species including proteins useful as cancer markers, where only the factor ‘mass’ was used in comparing serum proteins from a cancer patient with those of a normal human, and cancer-specific serum proteins are determined only by evaluating increased or reduced levels of a large number of serum proteins. Therefore, such a method of determining cancer-specific serum proteins using mass spectrometry is disadvantageous in terms of low accuracy in cancer diagnosis, as well as being not economical.

### DISCLOSURE OF THE INVENTION

Leading to the present invention, the intensive and thorough research for a method of screening diseases, which is simple and quick and which can be performed by ordinary persons, conducted by the present inventors aiming to overcome the above-mentioned problems, resulted in the finding that a disease-specific serum proteome standard can be obtained by transforming separated states of serum proteins on 2D-gels into images in order to facilitate distinction of modified proteins through separation of proteins contained in a serum sample in two dimensions, and mining the 2D-gel images using an image mining technique, and that such standards are useful in developing a simple and economical method and system of screening and classifying some specific types of cancer.

It is therefore an object of the present invention to provide a method of analyzing cancer using a proteome image mining technique, which facilitates early cancer detection, by collecting a plurality of serum proteomes from normal individuals and diseased individuals and transforming 2D-gel patterns of the serum proteome into two-dimensional images, producing serum proteome standards using an image mining technique and constructing a database consisting of the proteome standards, obtaining a 2D-gel image of the serum proteome from a subject organism, and comparing the image of the subject with a plurality of the serum proteome standards stored in the database.

It is another object of the present invention to provide a method of finding characteritic patterns of serum proteomes from diseased individuals, and distinguish them from those of normal individuals, by applying an image-mining tool to two-dimensional images of serum proteomes.

It is still another object of the present invention to provide a method and system of analyzing cancer using a proteome image-mining tool, which makes it possible to obtain precise analysis results by analyzing serum proteomes using the image-mining tool employing a genetic algorithm and a support vector machine, and to follow-up the progress and prognosis of disease states by a fuzzy rule-based classification step.

It is a further object of the present invention to provide cancer-specific screening biomarkers, that is, proteome patterns, which provide great influences in cancer detection when such a method or system is applied.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram of a system for cancer analysis according to the present invention;

FIG. 2 is a detailed block diagram of a proteome standard production means shown in FIG. 1;

FIG. 3 is a flowchart illustrating a method of cancer analysis according to the present invention;

FIG. 4 is a flowchart illustrating a method of producing the proteome standard shown in FIG. 3;

FIG. 5 is a photograph illustrating a two-dimensional image of a serum proteome;

FIG. 6 shows a process of producing a proteome standard after the input of serum proteome;

FIG. 7 shows an optimal parting plane determiined by a support vector machine;

FIG. 8 shows a training step of breast cancer detection using a support vector machine and a genetic algorithm;

FIG. 9 shows a testing step of breast cancer detection using a support vector machine and a genetic algorithm;

FIG. 10 shows a result of practical diagnosis of breast cancer in which 26 spots are used as the optimal feature data; and

FIG. 11 shows a result of practical usage of breast cancer screening in which 48 spots are used as the optimal feature data.

### BEST MODES FOR CARRYING OUT THE INVENTION

The present invention is directed to a method of analyzing cancer, comprising the steps of: transforming inputted serum proteomes from normal individuals and individuals having cancer into two-dimensional images, extracting feature data from the images, generating a proteome standard having a disease-specific proteome pattern by computing optimal features capable of distinguishing the two kinds of serum proteome from each of the feature data, and constructing a database consisting of the proteome standard; inputting a serum proteome from a subject of interest, transforming the serum proteome into a two-dimensional image and extracting feature data from the image; and comparing the structure of the serum proteome pattern of the subject with the proteome standard having a disease-specific proteome pattern and determining whether the serum proteome of the subject is normal or abnormal, that is, indicating the possible existence of cancer, or discriminating the type of cancer, based on the comprised results.

In addition, the present invention provides a system of diagnostic screening of cancer, comprising an input means for inputting serum proteome; a proteome standard production means for generating a proteome standard having a disease-specific proteome pattern by transforming received serum proteomes from a plurality of normal and diseased individuals into two-dimensional images and extracting features from the images, and extracting optimal features capable of distinguishing the two kinds of serum proteome from each of the feature data, and transforming a serum proteome of a subject into a two-dimensional image and extracting features from the image; a proteome comparison means for mapping the serum proteome pattern of the subject, extracted by the proteome standard production means, with the proteome standard pattern to determine similarities between the two patterns; a disease analysis means for estimating the serum proteome of the subject as ‘normal’ if the serum proteome pattern of the subject is similar to that of the normal individuals, and otherwise, as ‘having cancer’, based on the mapping results by the proteome comparison means; and an output means for outputting the analysis results by the disease analysis means.

Definition of Terms

If not defined elsewhere, technical and scientific terms used in the present specification have the meanings commonly understood by those skilled in the art.

The term “biomarker”, as used herein, refers to a polypeptide differentially present in serum samples from individuals having any disease, compared to that from normal individuals. Such a biomarker or biomarkers may comprise a single polypeptide or two or more polypeptides. In addition, the term “differentially present” means that a specific polypeptide in a serum sample from an individual having any disease has an increased or reduced expression level, or is newly present or absent, compared to a serum sample from a normal individual.

The term “proteome pattern”, as used herein, means a characteristic group or grouped form of polypeptides differentially present in a serum sample from an individual having any disease, compared to a serum sample from a normal individual. Typical examples of the proteome pattern include a group of serum proteins showing specific modification patterns in a specific disease, or a distribution pattern of the serum proteins in two dimensions. In addition, the term “disease-specific proteome pattern”, as used herein, refers to a group of serum proteins specifically appearing according to the kinds or types of diseases, or a grouped form of the serum proteins. Such a proteome pattern is used as a marker to detect diseases and identify the kinds or types of diseases using the method and system according to the present invention.

The term “feature data”, as used herein, refers to the data of a serum proteome, capable of distinguishing diseased states through comparison of serum proteomes from normal and diseased individuals. In detail the feature data includes data of spots corresponding to serum proteins specifically present on two-dimensional images of serum proteomes from diseased individuals. For example, the feature data may include a group (combination) of spots, mass of each of the spots, and/or an isoelectric point of each spot. In addition, the term “optimal feature data”, as used herein, refers to optimal data capable of specifically distinguishing diseases among the feature data. In detail, the optimal feature data includes optimal combinations among combinations of disease-specific spots.

The term “data mining”, as used herein, as a process of discovering useful correlations hidden in a large volume of data, refers to a process of identifying new data models derived from the data of the databases, which are previously unknown, and of extracting practicable information in the future and using the information for estimation That is, “data mining” means to discover valuable information by finding patterns and relations bidden in the data.

The term “genetic algorithm (GAY)”, as used herein, which deals with the ability of living individual to adapt to their environment by technologically modeling mechanisms associated with heredity and evolution of living individual, and refers to a technique of generating much better solutions by expressing possible solutions for problems as a data structure having a predetermined form and then gradually modifying the data structure. In more detail, the genetic algorithm is a kind of optimized search algorithm to seek an x value at a high speed to derive a maximum or minimum value of a function f(x) for a variable x defined within a certain range. The genetic algorithm typically comprises the steps of determining genetic types by performing coding work of transforming gene elements into symbol strings; determining an initial genetic group by generating a variety of individuals having different genetic elements from the genetic types determined at the step of determining genetic types; evaluating adaptability of individuals by computing adaptability of each individual by a predetermined method; determining survival distribution of individuals based on the adaptability determined at the step of evaluating adaptability; mating by exchanging genes between two chromosomes to generate new individuals; inducing mutagenesis by forcibly changing a portion of genes and thus maximizing diversity of a genetic group to generate individuals having much better solutions; and returning to the step of evaluating adaptability of each individual. Since the genetic algorithm finds solutions through mutual cooperation between a plurality of individuals by gene manipulation such as selection or mating, much better solutions are easily discovered. Also, the genetic algorithm has an advantage in that its operation is easy.

The term “support vector machine (SVM)”, as used herein, which is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for text classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase.

The method for disease analysis and system using the method according to the present invention will be described in detail with reference to the accompanying drawings. The method and system for disease analysis are useful for diagnosis of a variety of diseases, but in the present invention, their application to cancer diagnosis is illustrated.

Samples useful for standard generation and disease analysis include biological samples which may contain disease-specific polypeptides, which are exemplified by serum, urine, tears and saliva. In particular, serum proteomes from all individuals having genes are used as biological samples, but in the present invention, serum proteomes from humans are illustrated.

In the present invention, cancer means a pathogenic state caused by “uncontrolled cell growth”. Examples of cancer include breast cancer, ovarian cancer, stomach cancer, liver cancer, uterine cancer, lung cancer, large intestine cancer, pancreatic cancer and prostate cancer.

System for Disease Analysis Using an Image Mining Technique

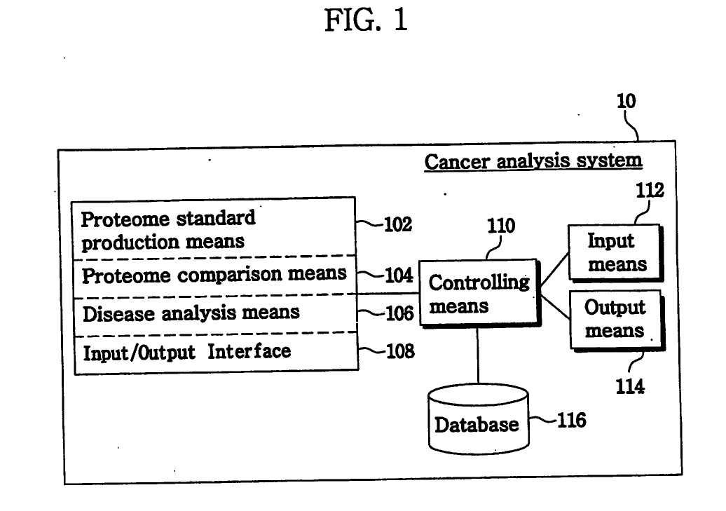

FIG. 1 is a block diagram of a system of analyzing cancer according to the present invention.

As shown in FIG. 1, a cancer analysis system 10 comprises a proteome standard production means 102, a proteome comparison means 104, a disease analysis means 106, an input/output interface 108, a controlling means 110, an input means 112, an output means 114 and a database 116.

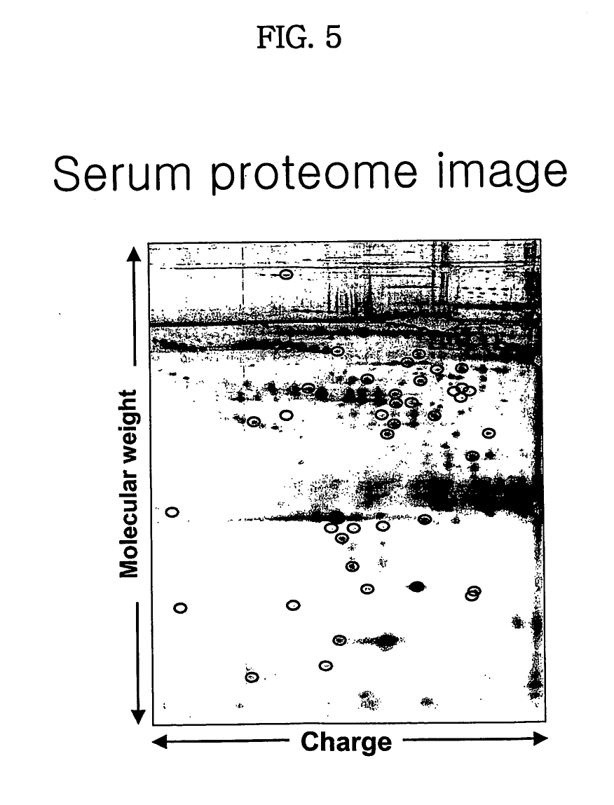

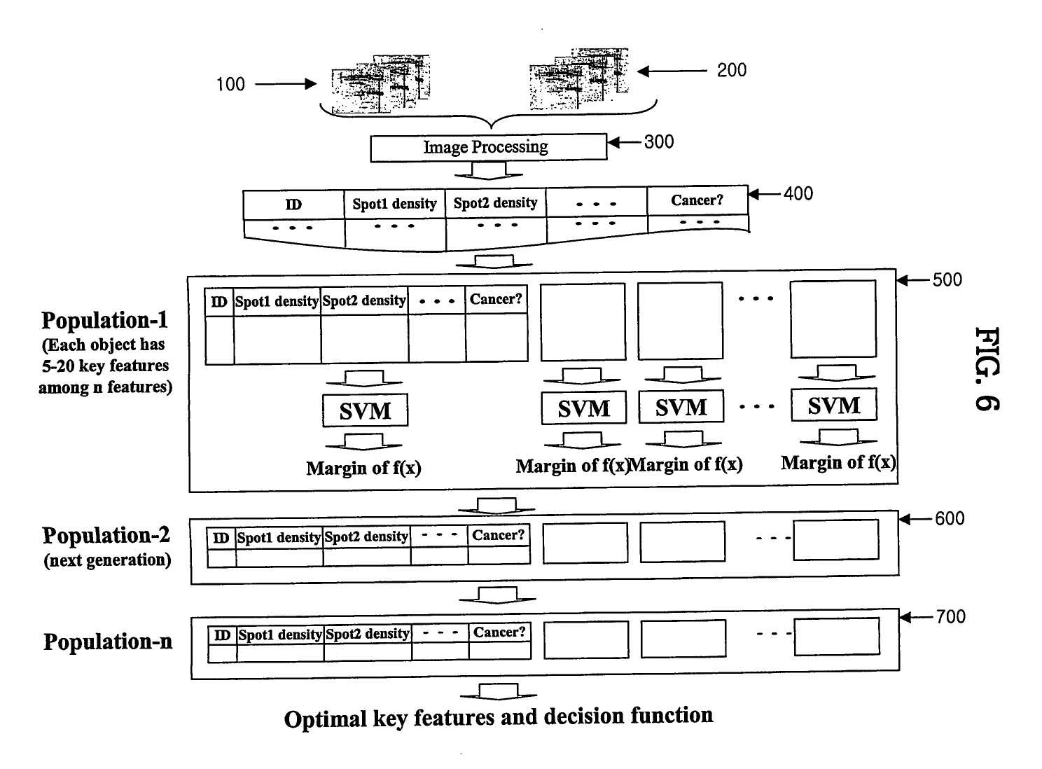

The proteome standard production means 102 receives serum proteome from N numbers (e.g., 20) of normal individuals and N numbers (e.g., 20) of diseased individuals through the input means 112, transforms the serum proteome into two-dimensional images (see, FIG. 5), extracts features, namely, specific spots, and distinguishes optimal feature data from the extracted feature data, while extracting and normalizing correlations between data consisting of spots in the two-dimensional images and storing the correlations in a database 116. For performance of such functions of the proteome standard production means 102, a genetic algorithm, a support vector machine and a fuzzy rule-based classification system are available, which will be described in detail, below. According to the operation of the proteome standard production means, features (intensity, size, etc.) of serum proteomes from individuals having cancer, different from a serum proteome standard of normal individuals, are discovered, and particularly, use of a fuzzy rule-based classification system allows to clarify the progression status and future prognosis of cancer and other diseases to be monitored.

In addition, the proteome standard production means 102 transforms serum proteome of a subject of interest as well as of normal and diseased individuals as standards into two-dimensional images, and extracts feature data from the images, and the resulting feature data are used in a process of analyzing whether a subject has a specific disease or not.

After the feature data of the serum proteome of a subject are extracted by the proteome standard production means 102, the proteome comparison means 104 determines whether a pattern of the serum proteome of a subject is similar to a pattem of a proteome standard stored in the database 116, through mapping the two patterns.

When a pattern of the serum proteome of a subject is similar to a pattern of serum proteome of normal individuals, the disease analysis means 106 determines the serum proteome of the subject as ‘normal’, and otherwise, as ‘having cancer’. The estimation results are outputted by the output means 114. Herein, when the subject is identified as having cancer, progression states and prognosis of cancer is predicted, and the predicted results are outputted. Also, in case that the subject does not have cancer at present, the probability of future cancer development can be predicted and outputted. To perform such functions, the proteome standard production means 102 should produce a standard data using a fuzzy rule-based classification system.

The input/output interface 108 is for connecting and integrating of the cancer analysis system 10 with an external apparatus, and the controlling means 110 controls overall operation of each functional means as described above.

The cancer analysis system 10 according to the present invention may further comprise a coding means (not shown in FIG. 1), thus allowing storage of personal information of normal individuals and individuals having cancer, who donate their serum proteome to be used as standards, and of personal information of subjects in the database 116 in a coded form.

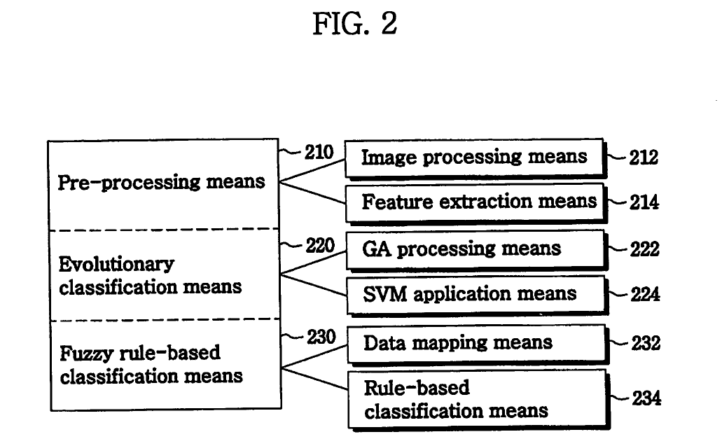

FIG. 2 is a detailed block diagram of the proteome standard production means 102 shown in FIG. 1.

The proteome standard production means 102 includes a pre-processing means 210 for obtaining meaningful feature data from the two-dimensional images of serum proteome; an evolutionary classification means 220 for identifing normality of serum proteome of a subject from the feature data obtained by the pre-processing means 210; and a fuzzy rule-based classification means 230 for estimation of more detailed states of the serum proteome of a subject from the feature data obtained by the pre-processing means 210 employing experimental knowledge, statistical tools, etc.

The pre-processing means 210 includes an image processing means 212 and a feature extraction means 214. The image processing means 212 performs general image processing works, including noise filtering, image enhancement, ortho-projection, edge detection and optimal thresholding, from the inputted two-dimensional images, while the feature extraction means 214 extracts basic features, namely, disease-specific spots from the image-processed two-dimensional images. Each feature extracted by the feature extraction means 214 is discriminated or labeled, thus producing feature data for spots.

The evolutionary classification means 220, which is a means for analyzing patterns of serum proteomes from normal or diseased individuals using the data obtained by the pre-processing means 210, comprises a GA (genetic algorithm) processing means 222 and a SVM (support vector mechanism) application means 224, and finds optimal combinations among combinations of disease-specific spots. The GA processing means 222 discriminates optimal features playing a critical role in classification among the feature data (disease-specific spots) extracted by the pre-processing means 210, while the SVM application means 224 estimates fidelity of the optimal feature data discriminated by the GA processing means 222 using decision functions and a classification error rate. Thus, possible spots of the next generation are produced, and through such an evolution method, optimal feature data and estimation functions according to the data can be generated. Herein, the estimation function used by the SVM application means 224 is a predetermined function.

Genetic algorithm (GA) is recently highlighted as a tool to be used in optimization. In accordance with the present invention, features of 5 to 20 information pieces can be effectively extracted using a genetic algorithm. The evolutionary classification means 220 extracts a plurality of features capable of easily and effectively screening cancer and other diseases. Later, by comparing features extracted from a test sample from a subject with a plurality of features as described above, whether the subject has cancer can be determined.

On the other hand, the fuzzy rule-based classification means 230 extracts processed information (medical history of a subject, medical history of the subject's family members etc.), which can be easily missed in the evolutionary classification step, for example, correlation between specific spots, through statistical and experimental methods, resulting in improvement of classification and recognition accuracy. The fuzzy rule-based classification means comprises a data mapping means 232 and a rule-based classification means 234. The data mapping means 232 computes correlations between spots from the two-dimensional images of serum proteome, classifies the computed features by a statistical technique, and quantifies the statistical inaccuracy using a fuzzy technique. The rule-based classification means 234 arranges and normalizes the results obtained by the data mapping means 232, thereby generating a final rule base. The fuzzy rule-based classification means 230 is not essential in the present invention, but its application in the present invention allows monitoring of progression status and prognosis of diseases through statistical and experimental methods by an expert system, as well as simple detection of cancer.

The method for disease analysis according to the present invention will be described in more detail, as follows. A method for cancer analysis according to the present invention comprises the steps of: generating a proteome standard having a disease-specific proteome pattern and constructing a database consisting of the proteome standard (traning step); and estimating whether a serum proteome of the subject is normal or indicative of a specific disease by extracting feature data from serum proteome of a subject of interest and comparing the feature data of the subject with the disease-specific proteome standard (testing step). In detail, the method for cancer analysis may be performed by a program stored in the memory and a processor connected to the memory, wherein the program can perform such a method. In addition, a program composed of instruction words executable by a digital processing device is typically realized, and a program for disease identification, comprising the evolutionary classification step and the fuzzy rule-based classification step according to the present invention may be stored in a recording medium readable by a digital processing device. Moreover, it will be apparent to those skilled in the art that the method for cancer analysis according to the present invention and each module for performance of the method can be realized in the from of a software, FPGA, ASIC, etc.

Production of a Proteome Standard Having a Disease-Specific Proteome Pattern (Training Step: S1)

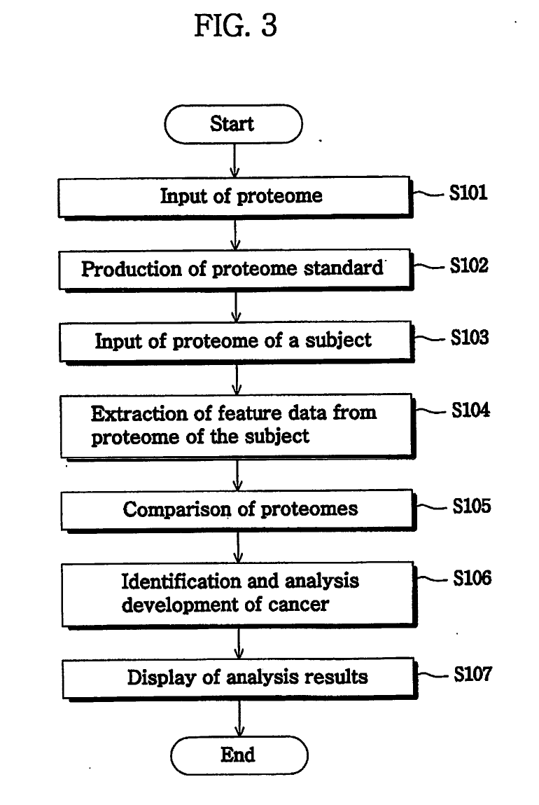

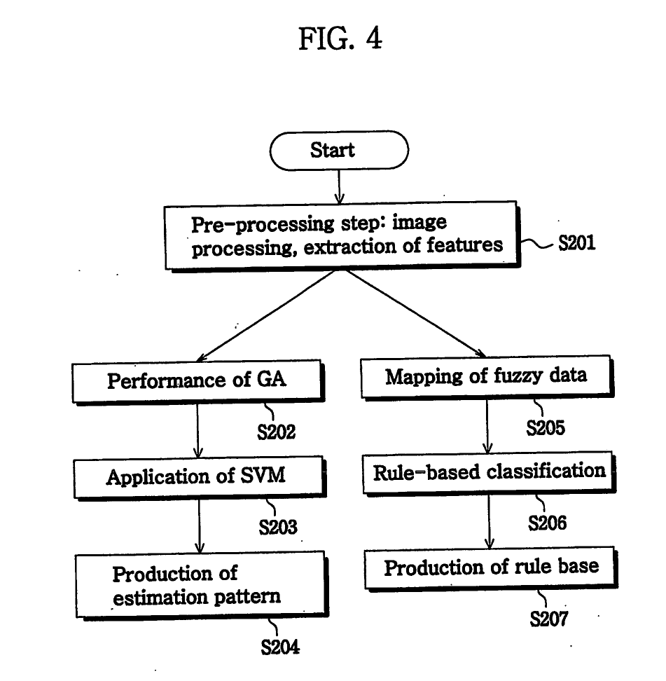

FIG. 3 is a flowchart illustrating the method of cancer analysis according to the present invention, while FIG. 4 is a flowchart illustrating a method of producing a proteome. standard shown in FIG. 3.

A disease-specific proteome standard of the present invention is generated by comparing serum proteomes from normal individuals with serum proteomes from diseased individuals and then finding a disease-specific proteome pattern.

To determine a serum proteome standard in the present invention, an image mining tool, for example, support vector machine, is used. Data mining is formed to discover useful correlation hidden in a large volume of data, and refers to a process of identifying new data models derived from the data of the databases, which are previously unknown, and extracting information useful in the future and using the information for estimation. That is, data mining means to discover valuable information by finding relations and patterns hidden in the data. Data mining can be applied for image analysis, which is a tool to extract patterns from digitalized pictures and used in diverse fields, including recognition of characters, medical diagnostics and the defense industry.

A method of discovering disease-specific proteome patterns includes the steps of receiving serum proteomes from normal and diseased individuals by the input means 112 (S101); and separating each of the serum proteomes on a 2D-gel, transforming each of the separated patterns into a two-dimensional image by the proteome standard production means 102, extracting disease-specific features (especially, cancer-specific features), finding optimal feature data among the extracted feature data to produce an optimal standard, and storing the result in a database(116)(S102). A process of producing a proteome standard will be described in more detail with reference to FIGS. 4 to 6, as follows.

The analysis of proteomes in the present invention may be performed by the conventional methods known to those skilled in the art, and preferably, by a 2D-gel analysis method. The 2D-gel analysis method used in the present invention is performed according to the conventional procedure in the art, in which proteins are primarily separated by their net charges (isoelectric focusing: IEF), and secondarily separated by their molecular weights (SDS-PAGE). In the present invention, serum proteins from normal individuals and serum proteins from diseased individuals are separated on 2D-gels.

FIG. 5 is a photograph illustrating a serum proteome image. As shown in FIG. 5, a separated pattern of serum proteins on a 2D-gel is transformed into a digitalized photograph to process the protein pattern on a 2D-gel into an analyzable form. Then, disease-specific features are extracted from the image information of the serum proteome, transformed into a digital information format, and stored. That is, specific features common in two-dimensional image information of serum proteomes from a plurality of normal and diseased individuals are extracted, and each data item (coordinate, molecular weight, isoelectric point, etc.) of the features are stored to construct a database. For example, a database may be generated by storing information (coordinate, molecular weight, isoelectric point, etc.) of intensity, size, etc. of differentially expressed spots, when comparing two-dimensional images of serum proteomes from individuals having a specific disease with two-dimensional images of serum proteomes from normal individuals. In addition, a database may be constructed by extracting common features between two-dimensional image information of serum proteomes from a plurality of diseased individuals, and storing each data item (coordinate, molecular weight, isoelectric point, etc.) of the features.

The disease-specific features mean specific spots having disease-specific intensity and size among spots in images obtained by separating serum proteomes by charge and molecular weight.

For example, through analysis of serum proteomes, molecular weight and acidity of a large number of proteins are evaluated, and a specific number is given to each of the proteins. Among the proteins, some proteins are extracted as cancer biomarkers capable of effectively detecting a specific cancer, thereby producing a cancer-specific proteome pattern. When analyzing a plurality of serum proteomes from breast cancer patients, a total of 67 specific spots are selected, which show features specific to breast cancer. The specific spots are listed in Table 1, below, in which their molecular weights and isoelectric points (pI) are indicated.

Optimal feature data is distinguished from the stored feature data, while correlations between data from spots in two-dimensional images are extracted and normalized to construct a database. For these, a genetic algorithm, a support vector machine (SVM and a fuzy rule-based classification system may be used. The resulting optimal feature data become proteome patterns of individuals having a specific disease, distinguishable from that of normal individuals, thus generating disease-specific proteome patterns.

In detail, each of various combinations of spots listed in Table 1, above, may give a breast cancer-specific proteome pattern. That is, a combination consisting of one or more spots selected from spots listed in Table 1 can be used as a breast cancer-specific pattern upon diagnostic screening of the breast cancer. Herein, to select one or more spots means one or more combinations of the total 67 spots, that is,

$${\sum }_{n = 1}^{67} {}_{67}^{}{C}{}_{n}^{} .$$

TABLE 1

| No. of spot | Molecular weight (kDa) | Isoelectric point (pI) |
|---|---|---|
| 106 | 20.2 | 4.60 |
| 204 | 27.4 | 4.57 |
| 406 | 52.8 | 4.58 |
| 1109 | 15.7 | 4.87 |
| 1303 | 41.0 | 4.82 |
| 1311 | 39.9 | 4.89 |
| 1318 | 38.4 | 4.99 |
| 2105 | 20.0 | 5.04 |
| 2114 | 25.8 | 5.22 |
| 2115 | 16.2 | 5.19 |
| 2204 | 32.9 | 5.07 |
| 2301 | 41.0 | 5.02 |
| 2309 | 46.4 | 5.13 |
| 2316 | 42.1 | 5.21 |
| 2318 | 44.8 | 5.21 |
| 2705 | 138.3 | 5.03 |
| 3104 | 17.8 | 5.23 |
| 3106 | 24.9 | 5.23 |
| 3110 | 22.8 | 5.29 |
| 3113 | 25.8 | 5.32 |
| 3119 | 21.0 | 5.39 |
| 3306 | 40.7 | 5.23 |
| 3402 | 51.2 | 5.22 |
| 3421 | 57.3 | 5.23 |
| 3514 | 62.7 | 5.38 |
| 4110 | 25.7 | 5.49 |
| 4123 | 17.7 | 5.50 |
| 4215 | 28.6 | 5.51 |
| 4302 | 39.4 | 5.42 |
| 4311 | 40.7 | 5.50 |
| 4313 | 37.3 | 5.53 |
| 4318 | 44.9 | 5.59 |
| 4319 | 42.9 | 5.59 |
| 4321 | 47.1 | 5.63 |
| 4401 | 48.2 | 5.40 |
| 4601 | 74.0 | 5.43 |
| 5108 | 26.2 | 5.76 |
| 5122 | 21.0 | 5.71 |
| 5303 | 42.9 | 5.71 |
| 5304 | 39.3 | 5.71 |
| 5305 | 40.9 | 5.71 |
| 5313 | 40.7 | 5.85 |
| 5314 | 41.8 | 5.88 |
| 5405 | 50.6 | 5.73 |
| 5406 | 56.2 | 5.75 |
| 5408 | 52.2 | 5.75 |
| 5409 | 47.7 | 5.76 |
| 5417 | 54.3 | 5.68 |
| 6113 | 20.5 | 6.05 |
| 6115 | 21.0 | 6.08 |
| 6204 | 35.5 | 5.92 |
| 6231 | 33.9 | 6.08 |
| 6302 | 42.5 | 5.89 |
| 6305 | 47.1 | 5.97 |
| 6306 | 39.4 | 5.98 |
| 6307 | 45.6 | 5.99 |
| 6308 | 37.7 | 5.99 |
| 6309 | 41.6 | 6.03 |
| 6310 | 46.3 | 6.04 |
| 6311 | 44.6 | 6.04 |
| 6312 | 45.6 | 6.08 |
| 6403 | 52.0 | 5.89 |
| 6418 | 53.1 | 6.05 |
| 7307 | 38.0 | 6.12 |
| 7312 | 42.7 | 6.14 |
| 7314 | 44.1 | 6.18 |

With reference to FIG. 4, to obtain meaningful feature data from two-dimensional images of serum proteomes, the proteome standard production step further comprises a pre-processing step (S201) and an evolutionary classification step (S202-S204). To enable to evaluate the progression status and future prognosis of diseases by statistical and experimental methods as well as simple detection of cancer, the process of producing a proteome standard may further comprise fuzzy rule-based classification steps (S205-S207).

In addition, the pre-processing step (S201) includes the steps of processing images and extracting features. At the image processing step, general image processing works, including noise filtering, image enhancement, ortho-projection and edge detection from the inputted two-dimensional images, are performed by the image processing means 212. At the feature extraction step, basic features in a spot form are extracted from the image-processed two-dimensional images by the feature extraction means 214. Each of the features extracted at the feature extraction step is discriminated or labeled, thus producing feature data for spots.

At the evolutionary classification step, patterns of serum proteomes from normal or diseased individuals are classified using the data obtained by the pre-processing step. The evolutionary classification step includes a GA (genetic algorithm) processing step (S202) and a SVM (support vector mechanism) application step (S205), as well as a step (S204) of extracting optimal feature data and estimation functions according to the data, which are discriminated at the GA processing step and the SVM application step. At the GA processing step (S202), spots having optimal features playing a critical role in classification of disease-specific spots are discriminated among feature data extracted by the GA processing means 222 at the pre-processing step. At the SVM application step (S203), fidelity of the optimal feature data discriminated at the GA processing step is estimated by the SVM application means 224 using decision functions and classification error rates. Thus, an alternative for spots of the next generation is produced, and through such an evolution method, optimal feature data and estimation functions according to the data can be generated(S204). Herein, the estimation functions used by the SVM application means 224 are predetermined functions.

FIG. 6 shows a process of producing a proteome standard from the pre-processing step to the evolutionary classification step. Using collection of serum proteome images (100) of diseased individuals and collection of serum proteome images (200) of normal individuals, image pre-processing is performed (300). Disease-specific spots are extracted from the proteome images of diseased and normal individuals, and disease-specific spots are determined according to their intensity and size and a database including features is constructed (400). A plurality of features extracted as described above have 5 or more feature spots, and preferably, 5 to 100 feature spots. Feature data (disease-specific spots) of the first generation are applied to a support vector machine (500), thereby producing optimal feature data and estimation functions according to the data. In addition, for serum proteome images in the second and N generations generated by inducing mating and mutagenesis of genes by a genetic algorithm, the same process as in the first generation is executed (600 and 700), thus giving final optimal features and estimation functions.

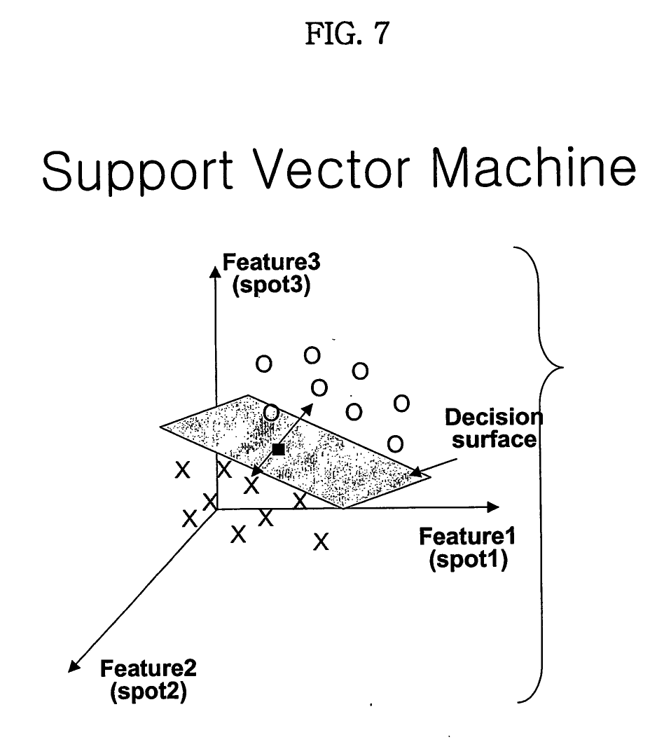

FIG. 7 shows an optimal parting plane determined by a support vector machine, in which an optimal interface, namely, optimal parting plane, is drawn by correlations among features from spot 1, spot 2 and spot 3, wherein the features are extracted from serum proteome images.

A fizzy rule-based classification step, which is a step for improving classification and recognition accuracy by extracting processed information, which can be easily missed in the evolutionary classification step, for example, correlations between specific spots, by statistical and experimental methods, comprises a data mapping step (S205), a rule-based classification step (S206) and a step of producing a rule base (S207) based on the two steps. At the data mapping step (S205), correlations between spots from two-dimensional images of serum proteome are computed by a data mapping means 232, the computed features are classified by a statistical technique, and statistical inaccuracy is quantified using a fuzzy technique. At the rule-based classification step (S206), the results obtained by the data mapping are arranged and normalized by a rule-based classification means 234, thereby generating a final rule base (S207). The fuzzy rule-based classification step is not essential in the present invention, but its application in the present invention allows monitoring the progression and prognosis of diseases through statistical and experimental methods by an expert system, as well as simple detection of cancer.

The process of producing a proteome standard according to the present invention, comprising the steps of extracting features (disease-specific spots) from image information of serum proteome from N numbers (e.g., 20) of normal individuals and N numbers (e.g., 20) of diseased individuals, and then producing a proteome standard by computing optimal features from feature data, may further include a step of estimating more detailed information of two-dimensional images of serum proteomes from subject individual by employing experimental data, a statistical method, etc.

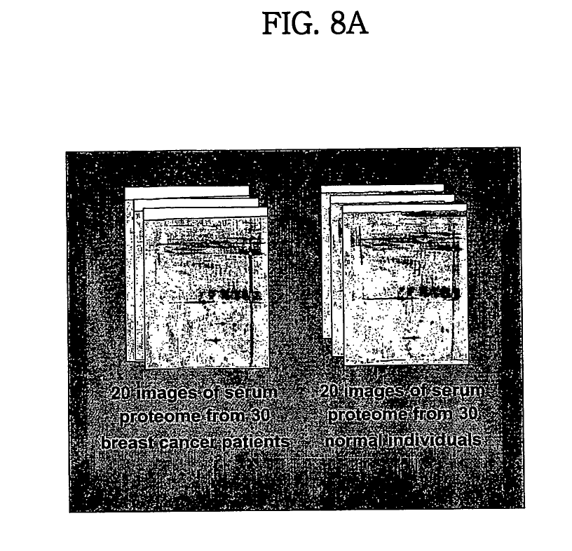

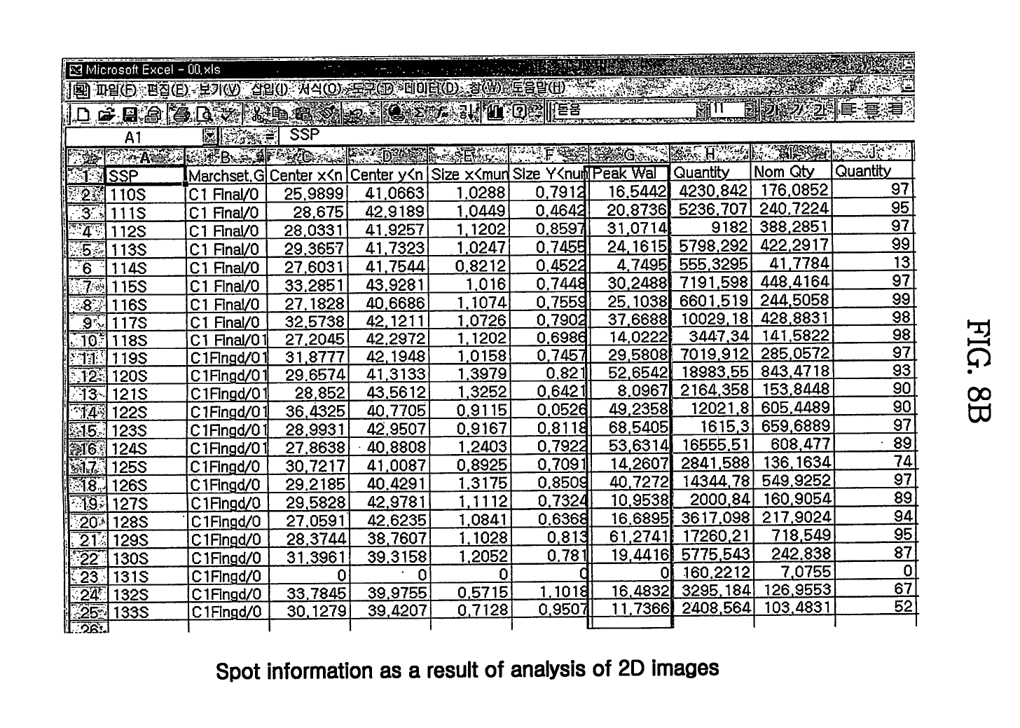

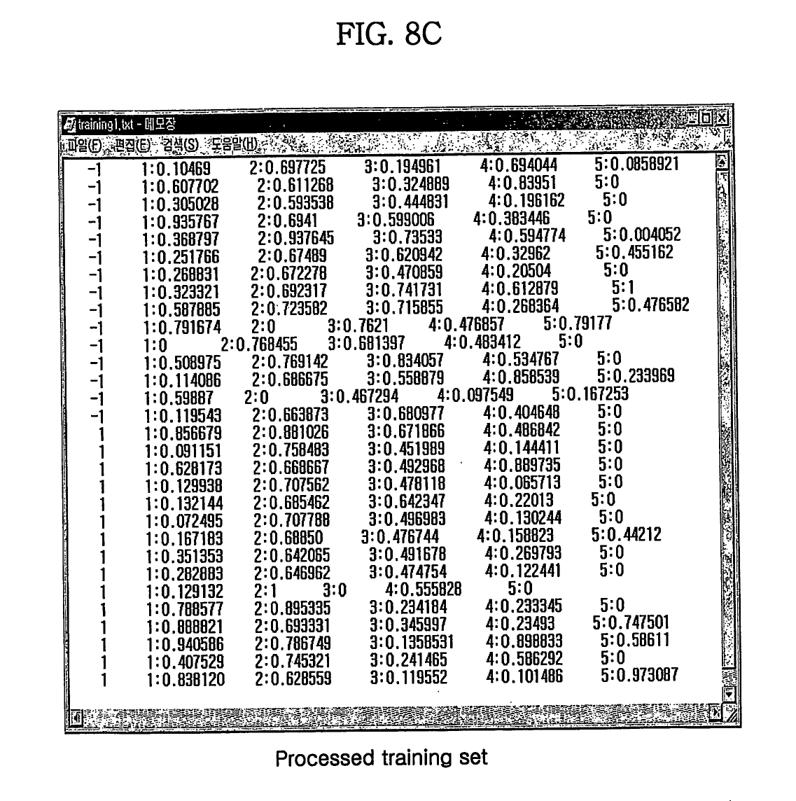

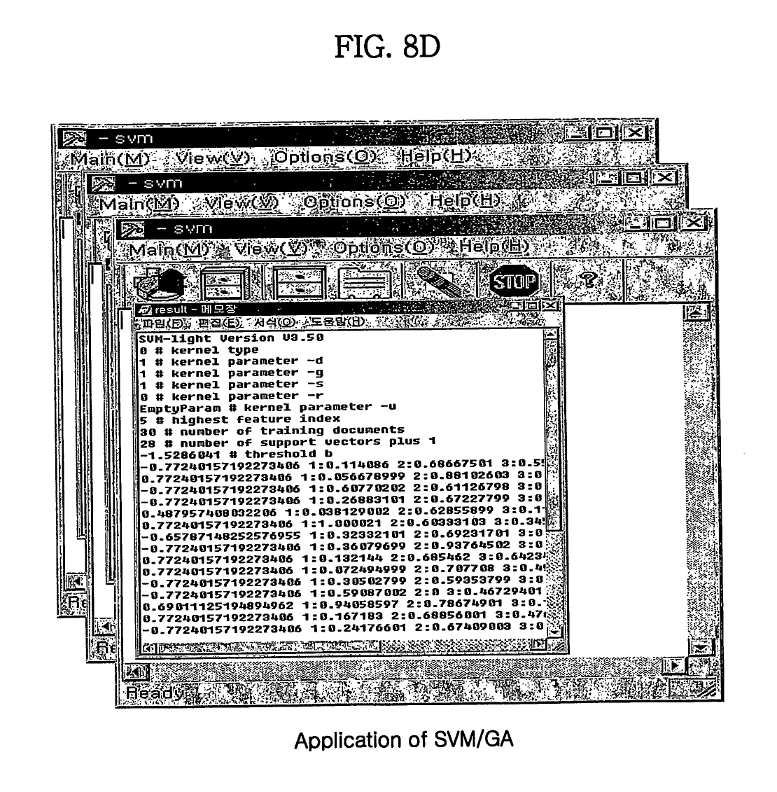

FIG. 8 shows an application of a process of producing a proteome standard having a disease-specific proteome pattern to diagnostic screening of the breast cancer (diagnosis) (training step). As shown in FIG. 8, through analysis of two-dimensional images of serum proteomes from 30 normal individuals and 30 individuals having a breast (specific) cancer, information of spots are collected and processed, and cancer-specific proteome patterns are searched using a support vector machine and a genetic algorithm.

Estimation of Development of a Specific Disease Through Comparison of Proteome of the Subject with a Disease-Specific Proteome Standard (Testing Step: S2)

After producing a standard by analyzing serum proteomes from normal and diseased individuals, as described above, a serum proteome of a subject of interest is inputted by the input means 112 (S103), and feature data are then extracted by the proteome standard production means 102 (S104). In more detail, serum proteome of a subject is separated on a 2D-gel according to the same method as in the image pre-processing step for production of a proteome standard, and the resulting 2D-gel image is transformed into a digital information format. Basic image processing works, including noise filtering, image enhancement, ortho-projection and edge detection, are performed for the two-dimensional images of a subject, and specific data as proteome patterns are then extracted. The resulting proteome patterns are used for comparison with the disease-specific proteome standard.

Estimation of whether a subject of interest has cancer through comparison of the proteome of the subject with a disease-specific proteome standard is achieved by performing a step of comparing the two proteomes (S105) and a step of determining whether the subject has cancer or not (S106). The results of analysis of cancers are displayed by the output means 114 (S107).

At the step of comparing proteomes (S105), the structure of a serum proteome pattern from a subject of interest is compared with the disease-specific proteome standard stored in the database 116 by the proteome comparison means 104, and whether serum proteome of the subject is normal or abnormal is analyzed by the disease analysis means 106. When more detailed states of serum proteome of the subject are stored at the training step using a fuzzy rule-based classification means employing experimental knowledge, a statistical method, etc., future prognosis as well as present states of serum proteome of the subject can be determined.

A pattern matching step is performed to screen the cancer, which may further comprise a fine classification step in the case that a fuzzy rule-based classification means is applied at the training step. At the pattern matching step, classification into “normal” or “having a disease” is performed using a support vector machine by applying features and estimation functions, extracted upon producing the proteome standard, to the pre-processed serum proteome of a subject of interest. In addition, at the fine classification step, fine information including correlations between spots are deduced by projecting the pre-processed serum proteome of a subject to a rule base produced at the fuzzy rule-based classification step.

The support vector machine (SVM, as defined above, comprises two steps: a training step and a testing step. At the training step, data vectors are inputted from a training set. In the present invention, the step of inputting results of pre-processing of serum proteome from N numbers of normal individuals and individuals having cancer corresponds to the training step. Then, the input data vectors from the training set are transformed into a multi-dimensional space, and parameters for support vectors and weights are determined. At the testing step, data vectors are inputted from a testing set, and the input vectors from the testing set are transformed into a multi-dimensional space by data matching. Then, a classification signal is produced from an optimal parting plane representing states of each input data vector. That is, whether the input data vectors from the testing set are normal or abnormal is determined.

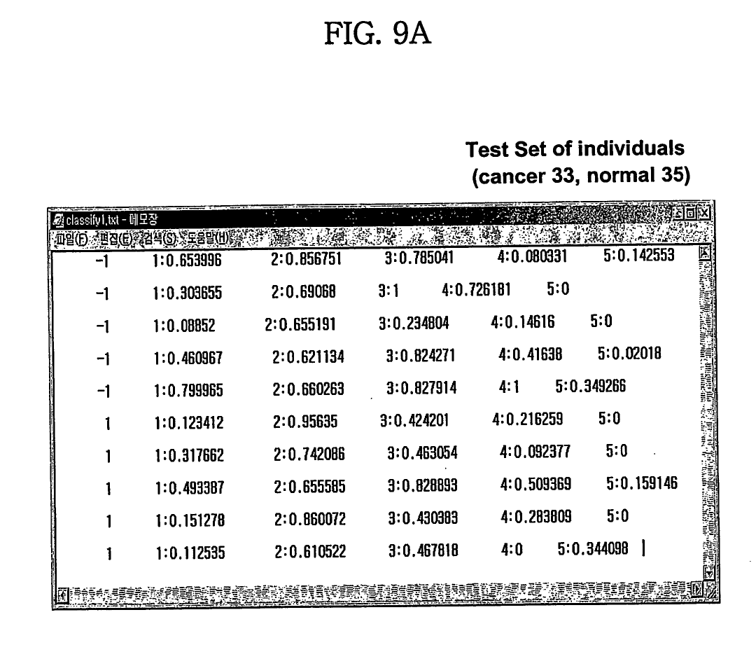

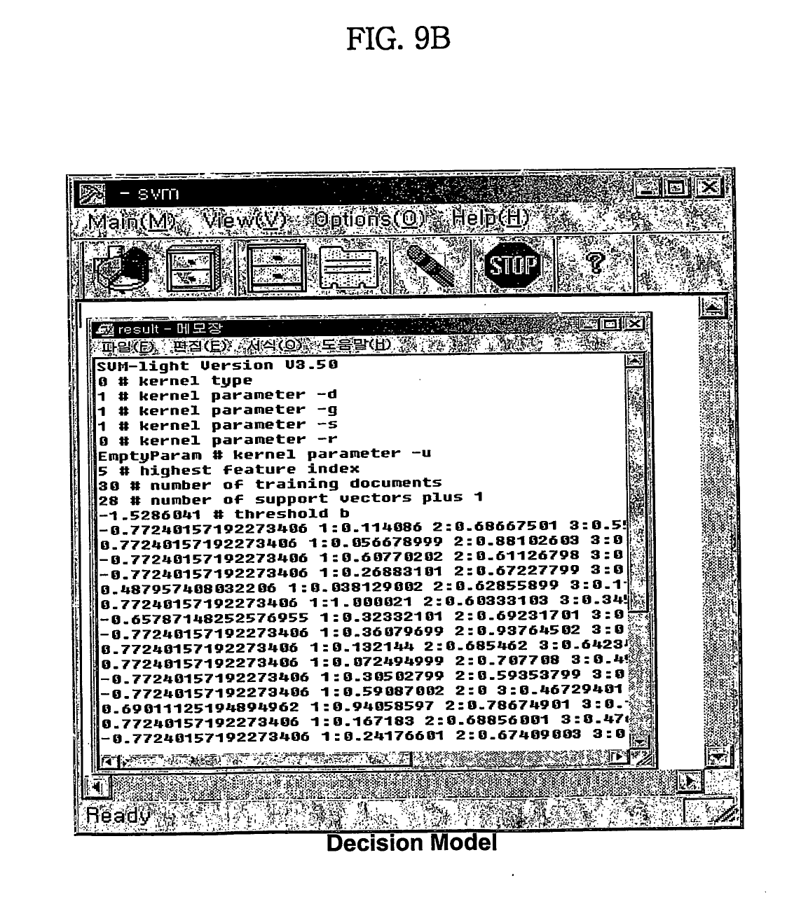

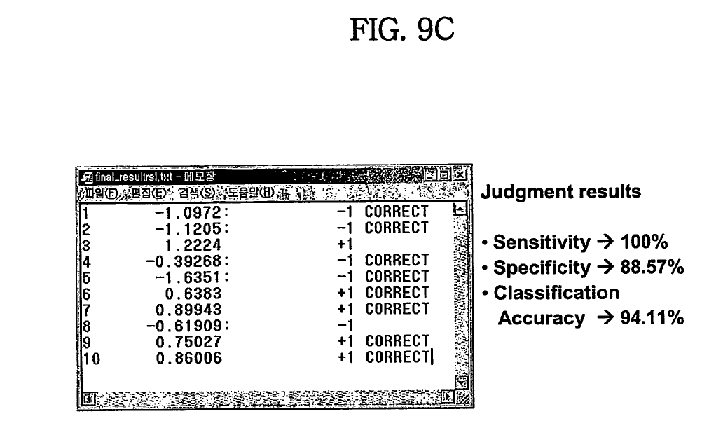

FIG. 9 shows a practical application of the step of estimating whether a subject has a disease through comparison of the proteome of the subject with a disease-specific proteome standard (testing step: S2). Based on decision models for breast cancer, produced at the training step (S1), a test set consisting of 33 cancer patients and 35 normal individuals was tested.

In a preferred embodiment of the present invention, serum proteomes of subjects of interest and analysis results are stored in the database 116, which are useful for later analysis of other proteomes.

In the following example, the system and method for disease analysis according to the present invention are applied to practical cancer screening.

### EXAMPLE 1

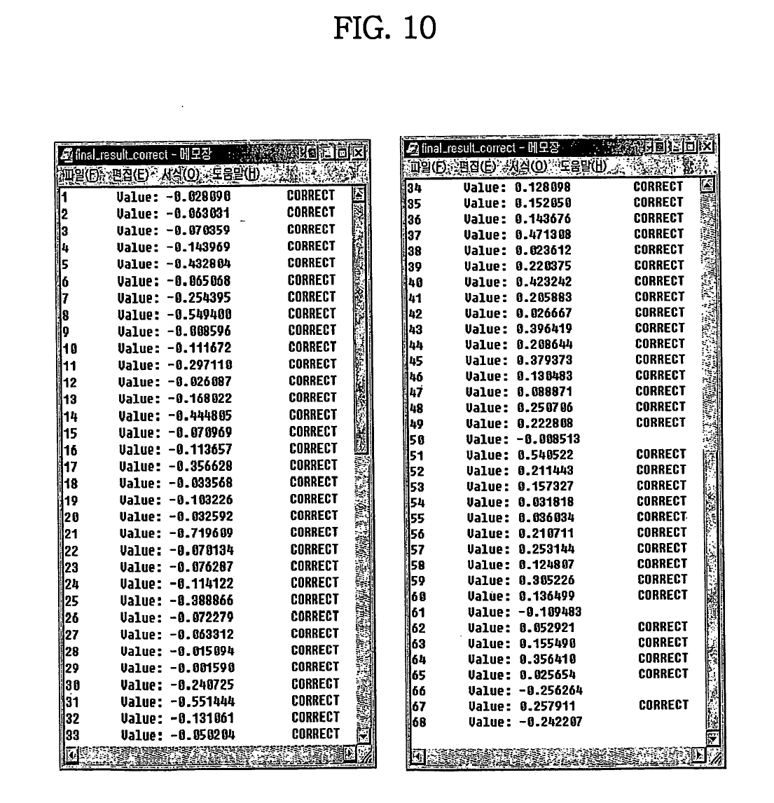

After training two-dimensional images of serum proteomes from 30 breast cancer patients and serum proteomes from 30 normal individuals, a test was performed for 33 cancer patients and 35 normal individuals. Such test through analysis of serum proteomes was found to have an accuracy of 94.11%, a sensitivity of 100% and a specificity of 88.57%. In this test, 26 spots were used as optical feature data of breast cancer. The 26 spots were selected from 67 breast cancer-specific spots listed in Table 1, above. The results are given in more detail in FIG. 10, in which accuracy means a degree of correctly estimating real breast cancer, sensitivity means rate of correctly identifying positives itself, and specificity means a degree of distinguishing breast cancer from other cancer diseases.

It will be apparent to one skilled in the art that various changes and modifications can be made in the present invention without departing from the spirit and scope of the present invention. It will be understood that the above example is described in an illustrative manner and is not to be constructed to limit the present invention. Therefore, it is to be understood that the scope of the present invention will be shown by the following claims rather than the above detailed description, and all modifications and variations of the present invention fall within the scope of the appended claims.

As described herein before, in accordance with the present invention, the system and method for disease analysis facilitates cancer screening by extracting features corresponding to disease-specific spots by applying an image mining technique to serum proteomes from normal and diseased individuals, constructing a database consisting of the features, and comparing the serum proteome of a subject of interest with proteome standards, thereby allowing early detection of cancer states. In addition, by introducing a fuzzy rule-based classification method, the system and method for disease analysis can monitor progression status and future prognosis of cancer diseases, thus making it possible to perform medical treatment suitable for pathologic states of patients.

1. A system of analyzing cancers using a proteome image mining tool, comprising:
input means for inputting serum proteome;
proteome standard production means for generating a proteome standard by transforming received serum proteome from a plurality of normal and diseased individuals into two-dimensional images and extracting spots as cancer-specific features from the images, and distinguishing optimal feature data among the extracted feature data, and transforming serum proteome of a subject into a two-dimensional image and extracting features from the image;
proteome comparison means for mapping the serum proteome pattern of the subject, extracted by the proteome standard production means, with the proteome standard pattern to determine similarity between the two patterns;
disease analysis means for estimating the serum proteome of the subject as ‘normal’ if the serum proteome pattern of the subject would be similar to that of the normal individuals, and otherwise, as ‘having cancer’, based on the mapping results by the proteome comparison means; and
output means for outputting the analysis results by the disease analysis means.

2. The system as set forth in claim 1, wherein the cancers are selected from the group consisting of breast cancer, ovarian cancer, stomach cancer, liver cancer, uterine cancer, lung cancer, large intestine cancer, pancreatic cancer, or prostate cancer.

3. The system as set forth in claim 1, wherein the proteome standard is one or more selected from spots listed in Table 1.

4. The system as set forth in any of claims 1 to 3, wherein the proteome standard production means distinguishes the feature data by extracting and normalizing correlations between spots comprising the proteome standard.

5. The system as set forth in any of claims 1 to 3, wherein the disease analysis means predicts progression status and future prognosis of cancer when the subject is identified as having cancer, and, in case that the subject does not have any cancer, predicts probability of cancer development.

6. The system as set forth in any of claims 1 to 3, wherein the system of analyzing cancers further comprises coding means for coding personal information of normal individuals and individuals having cancer, who donate their serum proteomes to be used as standards, and of personal information of subjects.

7. The system as set forth in any of claims 1 to 3, wherein the proteome standard production means comprises pre-processing means for obtaining meaningful feature data from the two-dimensional images of serum proteome, and evolutionary classification means for identifying normality of a serum proteome of a subject from the feature data obtained by said pre-processing means.

8. The system as set forth in claim 7, wherein the proteome standard production means further comprises fuzzy rule-based classification means for extracting correlations between spots contained in the serum proteome from the feature data obtained by the pre-processing means, and classifying the extracted correlations by a statistical method.

9. The system as set forth in claim 8, wherein the fuzzy rule-based classification means comprises data mapping means for computing correlations between spots from the two-dimensional images of serum proteome, classifying the computed features by a statistical technique, and quantifying statistical inaccuracy using a fuzzy technique; and rule-based classification means for arranging and normalizing the results obtained by said data mapping means, and thus generating a rule base.

10. The system as set forth in claim 7, wherein the pre-processing means comprises image processing means for performing general image processing works, including noise filtering, image enhancement, ortho-projection, edge detection and optimal thresholding, from the two-dimensional images of serum proteome, and feature extraction means for extracting features of spots from the image-processed two-dimensional images and labeling each of the features.

11. The system as set forth in claim 7, wherein the evolutionary classification means comprises genetic algorithm processing means for discriminating optimal feature data among the feature data extracted by the pre-processing means, and support vector mechanism application means for estimating fidelity of the optimal feature data discriminated by said genetic algorithm processing means using estimation functions and a classification error rate.

12. A method of analyzing cancer diseases using a proteome image mining tool, comprising the steps of:
transforming inputted serum proteomes from normal individuals and individuals having cancer into two-dimensional images, extracting feature data from the images, generating a proteome standard by computing optimal features from the feature data, and constructing a database consisting of the proteome standard (Step 1);
inputting a serum proteome from a subject of interest, transforming the serum proteome into a two-dimensional image and extracting feature data from the image (Step 2); and
comparing the structure of the serum proteome pattern of the subject with the proteome standard and determining whether the serum proteome of the subject is normal or abnormal, that is, indicative of cancer (Step 3).

13. The method as set forth in claim 12, wherein the cancer diseases are selected from the group consisting of breast cancer, ovarian cancer, stomach cancer, liver cancer, uterine cancer, lung cancer, large intestine cancer, pancreatic cancer, or prostate cancer.

14. The method as set forth in claim 12, wherein the proteome standard is one or more selected from spots listed in Table 1.

15. The method as set forth in any of claims 12 to 14, wherein the Step 1 further includes the steps of extracting correlations between spots contained in the serum proteome from the two-dimensional images of the serum proteome employing experimental knowledge and a statistical method, and classifying the extracted correlations by a statistical method.

16. The method as set forth in any of claims 12 to 14, wherein the Step 3 further includes a step of identifying present disease states and estimating a future prognosis of the disease by analysing serum proteome of the subject.

17. The method as set forth in any of claims 12 to 14, wherein the Step 3 of identifying the existence (development) of cancer includes:
a pattern matching step of classifying the serum proteome of the subject into “normal” or “having a disease” by applying features and estimation functions, extracted upon producing the proteome standard, to the serum proteome of the subject; and
a fine classification step of deducing fine information including correlations between spots, contained in the two-dimensional proteome images.

18. The method as set forth in any of claims 12 to 14, further comprising a step of constructing a database consisting of the serum proteome of the subject and analysis results thereof, wherein said step is performed after the Step 3.

19. The method as set forth in any of claims 12 to 14, wherein the Step 1 of producing a proteome standard comprises:
a pre-processing step including an image processing step of performing noise filtering, image enhancement, ortho-projection and edge detection from the two-dimensional proteome images, and a feature extraction step of extracting basic features in spot form the image-processed two-dimensional images and producing feature data by labeling each of the extracted features; and
an evolutionary classification step of performing a genetic algorithm to discriminate optimal features playing a critical role in classification among the feature data extracted at the pre-processing step, and extracting optimal feature data and estimation functions by estimating fidelity of the optimal feature data discriminated by the genetic algorithm by a support vector machine using estimation functions and classification error rates.

20. The method as set forth in claim 19, wherein the Step 1 of producing a proteome standard further comprises:
a fuzzy data mapping step of computing correlations between spots from the two-dimensional images of serum proteomes obtained at the pre-processing step, and classifying the computed features by a statistical method, and quantifying statistical inaccuracy using a fuzzy technique; and
a rule-based classification step of arranging and normalizing the results obtained at the data mapping step, and thus generating a final rule base.

21. (A) A biomarkers or biomarkers for diagnosis of cancers, comprising a proteome pattern, wherein said proteome pattern is one or more selected from spots listed in Table 1.

22. The biomarker or biomarkers as set forth in claim 21, wherein the cancers are selected from the group consisting of breast cancer, ovarian cancer, stomach cancer, liver cancer, uterine cancer, lung cancer, large intestine cancer, pancreatic cancer, or prostate cancer.
